(19) United States
(12) Patent Application Publication
(10) Pub. No.: US 2006/0116645 A1
(43) Pub. Date: Jun. 1, 2006

(54) Syringe

(76) Inventors: Paul Scott Whitfield, Washington (GB); Nicholas James Tyrrell, Knaphill (GB)

Correspondence Address: Gregory J. Lavorgna, Esquire;Drinker Biddle & Reath LLP, One Logan Square, 18th & Cherry Streets, Philadelphia, PA, 19103, US

(21) Appl. No.: 11/171,770

(22) Filed: Jun. 30, 2005

(30) Foreign Application Priority Data
Jul. 7, 2004 (GB) 0415195.7

(51) Int. Cl. A61M 5/00

(52) U.S. Cl. 604/187

(57) ABSTRACT

The present invention relates to syringes. The syringe of the present invention is provided with a plastics material body moulded around a needle. The syringe provides particular advantage being used for the injection of local analgesia for dentistry or surgical procedures. The syringe of the present invention is intended to be a single use, disposable syringe to help combat the problems of needle stick injury.

### FIELD OF THE INVENTION

The present invention relates to syringes.

### BACKGROUND OF THE INVENTION

“Needle Stick Injury” is a problem associated with the use of syringes for medical purposes. It occurs when the person administering treatment to a patient with such a syringe is inadvertently “stuck” by the needle after it has been used on the patient.

Such injuries can transmit infections from patient to user; including HIV, Hepatitis A, Hepatitis B, Hepatitis C and new variant CJD.

Some state of the art syringes are reusable, and include a removable needle which fixes to the syringe, usually either by a screw thread or a bayonet fixing. This is particularly common with “dental” syringes which are of slim shape and use narrow glass or plastic vials of active material. Although the syringes themselves are reused, the needles are not and are thrown away after use into a specialised disposal unit, referred to as a “sharps” container or bin, for careful disposal.

Needle Stick Injury is often likely to occur when the needle is being removed from the syringe for disposal in such a sharps box.

Other known syringes are disposable but are not adapted for use with pre-filled vials. Disposbale syringes in common are supplied without needles, which must be attached by the user and are normally detached after use for disposal in a sharps container.

### SUMMARY OF THE INVENTION

According to the present invention there is provided a syringe comprising a body and a needle, the body being moulded around the needle.

Preferably the body is formed from a plastics material. The plastics material may be ABS (Acrylonitrile-Butadiene-Styrene) or other such material suitable for the moulding process.

Preferably the syringe further includes a chamber within the body adapted to receive a vial of anaesthetic, drug or other substance for injecting into a patient.

Preferably a portion of the needle projects into the chamber. The portion of the needle within the chamber punctures the seal of a vial when placed within said chamber, to allow the contents of the vial to be injected through the needle and into a patient.

Preferably there is also provided a plunger co-operable with the body and chamber, and in use is co-operable with a vial stopper of a vial.

Preferably the plunger is provided with a finger grip at one end and at the distal end there is provided a plunger tip, the plunger tip being adapted to engage with a vial stopper. In this way the plunger may be used to aspirate with the syringe in addition to injecting.

### BRIEF DESCRIPTION OF THE DRAWINGS

An embodiment of the present invention will now be described, by way of example only, with reference to the following drawings, in which:

FIG. 1 is a perspective view of a syringe according to the present invention;

FIG. 2 is a perspective view of an aspirating plunger of the syringe of FIG. 1;

FIG. 3 is a detail view of the tip of the plunger of FIG. 2;

FIG. 4 is a perspective view of a non-aspirating plunger compatible with the syringe of FIG. 1;

FIG. 5 is a perspective view of the needle of the syringe of FIG. 1;

FIG. 6 is a perspective view of a breech plug of the syringe of FIG. 1;

FIG. 7 is a detail view of a vial of anaesthetic for use in the syringe of FIG. 1; and

FIG. 8 is a perspective view in section of a stopper of the vial of FIG. 7.

### DETAILED DESCRIPTION OF THE PREFERRED EMBODIMENT

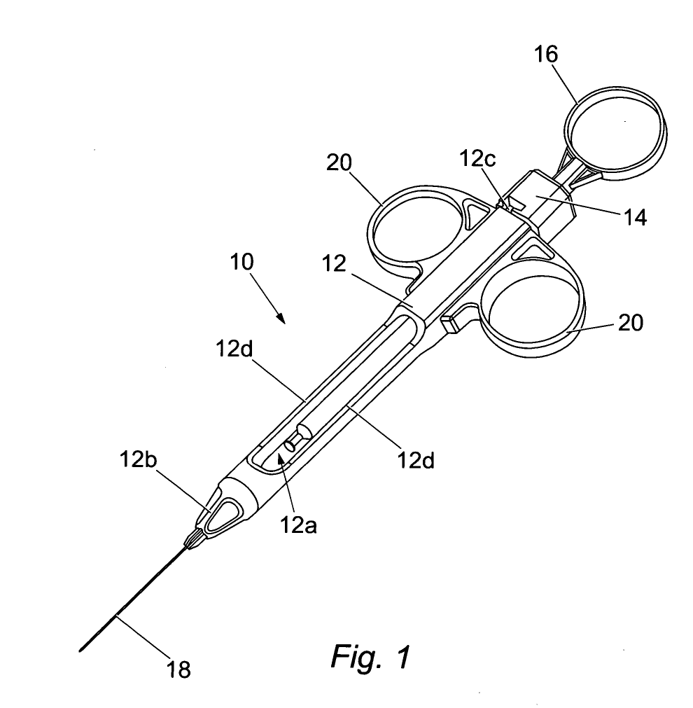

Referring to the drawings and initially to FIG. 1 there is shown a syringe according to the present invention referred to by the reference number 10.

The syringe 10 comprises a body 12, a breech plug 14 and a plunger 16. Integral to the body 12 and extending from one end of it is a hollow needle 18.

The body 12 is formed from ABS (Acrylonitrile-Butadiene-Styrene) resin, which is moulded to form the shape. The needle 18 is moulded within the body 12 extending between a central vial-holding chamber 12a through the nose 12b of the body 12 to the exterior of the body 12.

The vial-holding chamber 12a extends within the body 12 for the majority of the body's length and is divided into two sections. The first section, nearest the needle 18, is partially open so that in use the user can easily monitor the level of a substance within a vial placed in the chamber 12a. For that purpose, the two legs 12d which define the first section of the chamber 12a may include a graduated scale.

The second section is located at the opposite side of the first section to the needle 18, and is a simple bore through the body 12.

Two finger grips 20 are provided on the body 12, adjacent the second section of the chamber 12a. The finger grips 20 are formed as loop-shaped lugs on either side of the body 12. As the finger grips 20 are formed as loops, this aids the user in keeping a firm grip on the syringe 10.

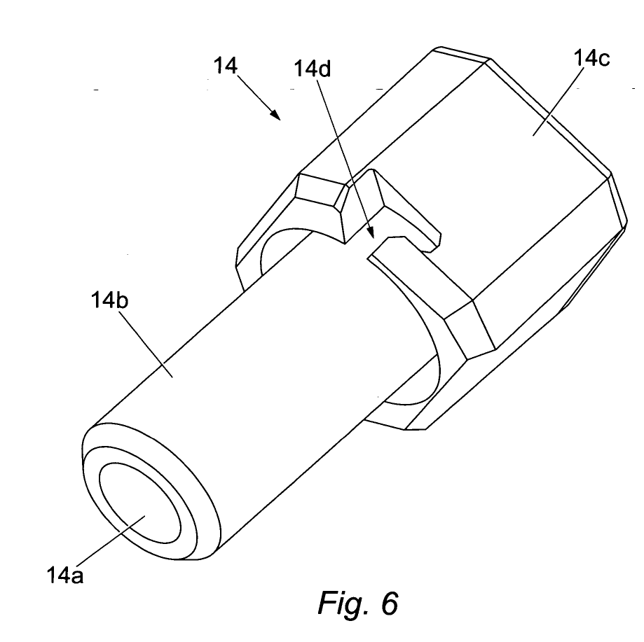

A breech plug 14 is also provided which fits into an initial portion of the second section of the chamber 12a. Detail of the breech plug 14 is shown in FIG. 6.

The breech plug 14 is also formed from ABS and includes a central bore 14a running through it. The breech plug 14 has a circular cross-section portion 14b that fits into the initial portion of the second section of the chamber 12a. The breech plug 14 also has a square cross-section portion 14c. The central bore 14a runs through both of these sections. In this embodiment, the body 12 and breech plug 14 have co-operating locking means to lock the breech plug 14 securely to the body 12 when the syringe 10 is in use, but permit the breech plug 14 to be removed from the body 12 when necessary. The locking means is provided by a simple projection 12c and L-shaped slot 14d, the projection 12c being provided on the body 12 with the L-shaped slot 14d being provided on the square cross section portion 14c. It will be understood that the locking means may be omitted without departing from the scope of the present invention.

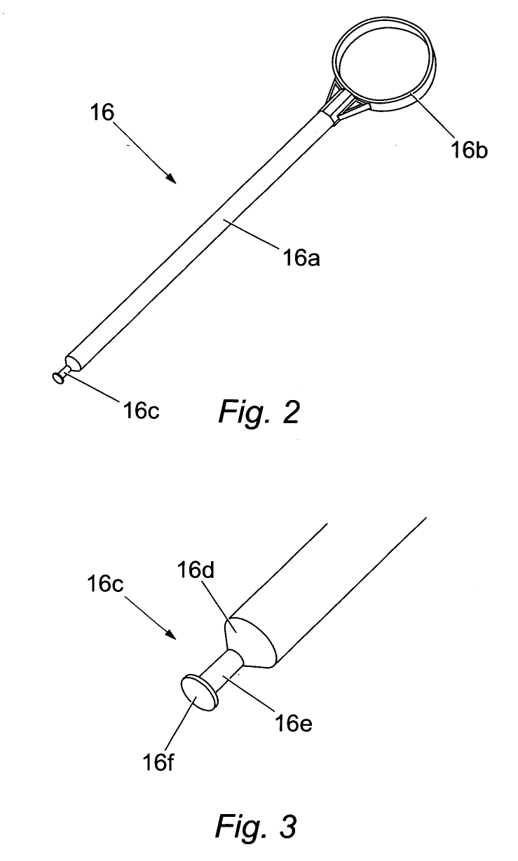

An aspirating plunger 16 is also provided, detail of which is shown in FIG. 2. The aspirating plunger 16 comprises a central stem 16a with a finger grip 16b provided at one end of the stem 16a. The finger grip 16b is also formed as a loop.

At the other end of the stem 16a from the finger grip 16b there is provided a vial-engaging tip 16c, shown in greater detail in FIG. 3.

The vial-engaging tip 16c is formed from three sections. The first section 16d is frusto-conical in shape, and tapers from the diameter of the stem 16a to a lesser diameter. The second section 16e follows on from the first section 16d and is a short cylindrical section which leads to the third section 16f, a flange of greater diameter than the second section 16e but of lesser diameter than the stem 16a.

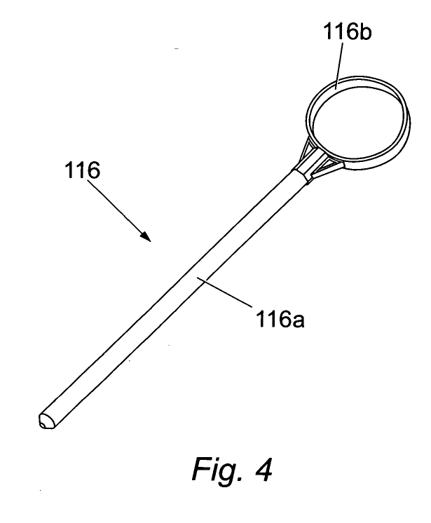

A non-aspirating plunger 116 is shown in FIG. 4, compatible with the syringe 10. This non-aspirating plunger 116 differs from the plunger 16 in that there is no vial-engaging tip 16c.

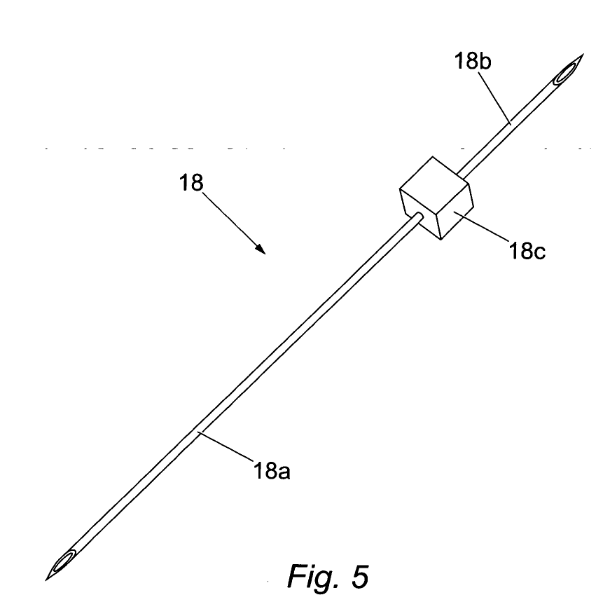

Referring to FIG. 5 there is shown detail of the hollow needle 18. The hollow needle 18 includes a long needle section 18a, a short needle section 18b and a cube section 18c.

When the needle 18 is moulded within the body 12, the long needle section 18a projects out from the body 12 for insertion into a patient. The short needle section 18b projects into the vial-holding chamber 12a. The cube section 18c is located within the nose 12b of the body 12. The shape of the cube section 18c assists the fixing of the needle 18 in place within the body 12 and impedes movement of the needle 18 within the moulded body 12.

In use, the user removes the breech plug 14 by twisting it so that the projection 12c is disengaged from the L-shaped slot 14d. The breech plug 14 can then be removed from the body 12 along with the plunger 16.

A vial 28 is inserted into the vial-holding chamber 12a. The sealed lid end (not shown) of the vial 28 is inserted first and is pushed through into the chamber 12a. The breech plug 14 is reattached to the body 12 and locked into place. The dimensions of the breech plug 14 and the action of reattaching forces the short needle section 18b to penetrate the sealed lid of the vial 28.

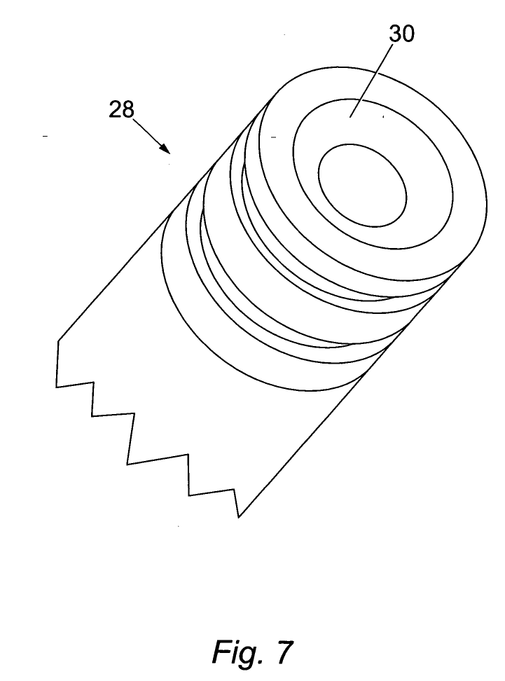

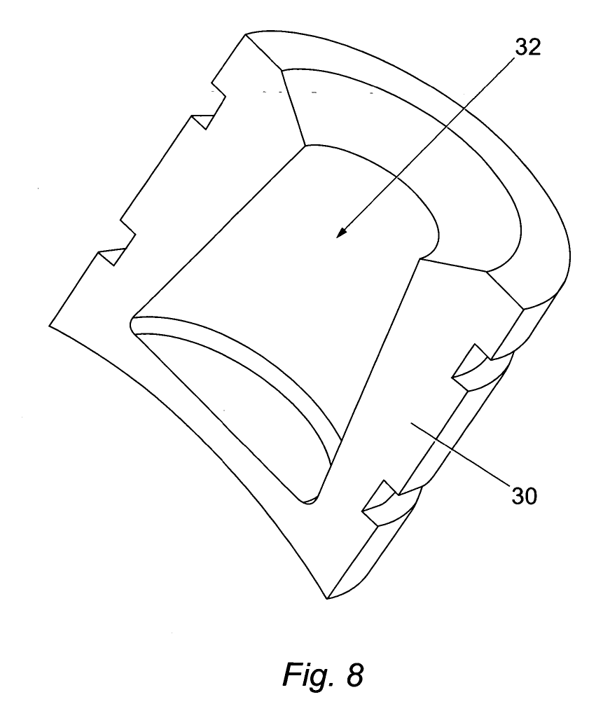

A plunger 16 or 116 can be fed through the breech plug 14 to engage a vial stopper 30 (see FIGS. 7 and 8). The vial stopper 30 depicted in FIGS. 7 and 8 has a conical bore 32 within it. Detail of the conical bore 32 can be seen in FIG. 8.

With the aspirating plunger 16, the plunger tip 16c is inserted into the conical bore 32. The flange 16f is slightly wider than the initial section of the conical bore 32, but since the vial stopper 30 is formed from a resilient material, the user can force the flange 16f into the conical bore 32. Once the plunger tip 16c is engaged with the vial plunger 30, the user is then able to inject by forcing the vial plunger 30 toward the nose 12b of the body 12 or can aspirate by pulling the vial plunger 30 away from the nose 12b. The ability to aspirate as well as inject allows the user to ensure that the long needle section 18a is correctly placed within the patient. If, for example, the syringe 10 is being used to inject a local anaesthetic into a patient it can be dangerous to inject a local anaesthetic into a blood vessel as this can cause bradycardia, slowing of the heart rate.

For situations where the ability to aspirate is not needed, a non-aspirating plunger 116 can be used instead of a plunger 16. The non-aspirating plunger 116 does not engage the vial stopper 30, but simply pushes against it, forcing it toward the needle 18 in use. Drawing the non-aspirating plunger 116 out of the vial chamber 12a does not aspirate, since the non-aspirating plunger 116 does not draw the vial stopper 30 back along the vial 28 from the needle 18.

Since the syringe 10 is made from a relatively inexpensive material, the present invention finds particular advantage as a single use or single patient (i.e. used several times but only with the one patient) device. After use, the entire syringe 10 can be disposed of without the user having to remove the needle 18 from the body 12.

The embodiment of present invention described herein also provides particular advantage when used for the injection of local analgesia for dentistry or cervical procedures, such as LLETZ (Large Loop Excision of the Transformation Zone), cold coagulation, etc, particularly due to its relatively slim profile (the body is approximately 10 mm in diameter).

In prior art metal syringes where the syringe body is reprocessed after use, there is a tendency for the syringe to wear and slightly deform. In syringes that have been used many times, this can lead to a vial breaking within the vial chamber because of misalignment between plunger and body. The relatively short lifetime of the present invention mitigates this tendency.

Modifications are envisaged to the present invention which do not depart from its scope. In addition to the graduated scale on the legs 12d, a collar made of rubber, foam or other suitable material may be provided in the vial chamber 12a to help locate the vial within the vial chamber.

It is to be understood that the present invention is not limited to the embodiment described herein, but may be further modified in both construction and detail.

1. A syringe comprising a body and a needle, the body being moulded around the needle.

2. A syringe according to claim 1 wherein the needle includes a thickened portion around a portion of its outer surface.

3. A syringe according to claim 2 wherein the thickened portion is generally cube-shaped

4. A syringe according to claim 1 wherein the body is formed from a plastics material.

5. A syringe according to claim 1 further including a chamber within the body adapted to receive a vial of anaesthetic, drug or other substance for injecting into a patient.

6. A syringe according to claim 1 wherein a portion of the needle projects into the chamber

7. A syringe according to claim 1 wherein there is also provided a plunger co-operable with the body and chamber, and in use is co-operable with a vial stopper of a vial.

8. A syringe according to claim 7 wherein the plunger is provided with a finger grip at one end and at the distal end there is provided a plunger tip, the plunger tip being adapted to engage with a vial stopper.
